(19) United States
(12) Patent Application Publication
(10) Pub. No.: US 2005/0130473 A1
(43) Pub. Date: Jun. 16, 2005

(54) Cleaning and disinfecting machine

(75) Inventor: Karl Heinze Annecke, Bensheim (DE)

Correspondence Address: SENNIGER POWERS LEAVITT AND ROEDEL, ONE METROPOLITAN SQUARE, 16TH FLOOR, ST LOUIS, MO, 63102, US

(73) Assignee: BHT Hygienetechnik GmbH, Gersthofen (DE)

(21) Appl. No.: 11/013,134

(22) Filed: Dec. 15, 2004

(30) Foreign Application Priority Data
Dec. 15, 2003 (DE) 103 58 999.6

(51) Int. Cl. H01R 13/44

(52) U.S. Cl. 439/135

(57) ABSTRACT

A cleaning and disinfecting machine has at least two exchangeable containers for different liquids. Liquids are sucked from the containers with suction lances having an electrical switch connected to a machine controller via electrical lines. The lances, containers, and controller are interrelated in a manner which prevents mix-up of exchangeable containers with respect to their respective lances.

### CROSS REFERENCE

This application claims priority from German application 103 58 999.6, filed Dec. 15, 2003, the entire disclosure of which is expressly incorporated herein by reference.

### FIELD OF THE INVENTION

The present invention relates generally to cleaning and disinfecting machines and more particularly to a machine for cleaning and disinfecting medical instruments.

### BACKGROUND OF THE INVENTION

Machines used to clean and disinfect medical instruments employ different cleaning and disinfecting liquids supplied in exchangeable containers. By means of suction lances, the appropriate liquid is sucked from the containers in a program-controlled way and supplied to the washing chamber of the machine. Such a machine has been sold for a long time by the applicant under the designation “INNOVA.”

Despite different color markings on the containers, the containers are sometimes mixed up and connected to the wrong suction lance, with the result that the medical instruments, e.g., endoscopes, are not cleaned and disinfected correctly. For example, one case was identified in which two containers with cleaning liquid were connected and thus no container had disinfecting liquid, so that an endoscope was cleaned multiple times but not disinfected.

To prevent mix-ups, it would be conceivable to provide containers with different feed openings for suction lances and to attach different suction lances to the machine, so that a suction lance could only be inserted into the “correct” container. But this would have the serious disadvantage that the entire container production would have to be converted, which would lead to considerable cost increases.

Thus, there is a need for a cleaning and disinfecting machine of the type described above that substantially prevents mix-ups of the exchangeable containers.

### SUMMARY OF THE INVENTION

In one aspect the invention is directed to a cleaning and disinfecting machine of the type having multiple containers for containing liquid and suction lances therefor. The machine has a first exchangeable container containing a first liquid and a second exchangeable container containing a second liquid, the second liquid being different from the first liquid. It also has a first suction lance operable to suck the first liquid out of the first container and a second suction lance operable to suck the second liquid out of the second container, the first suction lance comprising a first suction lance electrical switch for connection to a machine controller via a first electrical line and the second suction lance comprising a second suction lance electrical switch for connection to the machine controller via a second electrical line. There is a first electrical connector associated with the first exchangeable container and the first suction lance, the first electrical connector having a first component of the first electrical connector being connected to the first container, a second component of the first electrical connector being connected to the first suction lance electrical switch, and a first electrical connector electrical line completing an electrical connection of the first suction lance electrical switch and the controller when the first and second components of the first electrical connector are coupled together. There is a second electrical connector associated with the second exchangeable container and the second suction lance, the second electrical connector having a first component of the second electrical connector being connected to the second container, a second component of the second electrical connector being connected to the second suction lance electrical switch, and a second electrical connector electrical line completing an electrical connection of the second suction lance electrical switch and the controller when the components of the second electrical connector are coupled together.

In one embodiment, the component of the first connector that is connected to the first container is incompatible with the component of the second connector that is connected to the second suction lance and the component of the second connector that is connected to the second container is incompatible with the component of the first connector that is connected to the first suction lance.

In one aspect of the invention, the machine makes use of the electrical float switch, which is already present in the typical suction lance and which reports to the machine controller whether fluid is still in the container, for identifying the container in conjunction with a plug connector. Depending on the fluid in the container, a different plug connector is used which is not compatible with other plug connectors. One part of the plug connector is assigned to each suction lance and the other part to the corresponding container. The first part of the plug connector assigned to the container is connected rigidly to the container, e.g., by means of an electrical jumper line. The second part is connected to an electrical line leading from the float switch to the machine controller. The plug connector interrupts at least one conductor of the electrical line from the float switch to the controller as long as the plug connector is not coupled. The machine controller then obtains an error signal and does not start the cleaning and disinfecting process.

Thus, an artificially produced interrupt, which can be rescinded by the electrical jumper line acting as an electrical bridge, is created at the plug connector.

If the machine concerns only two containers with different liquids, a plug can be attached to one suction lance and a socket on the associated container and a socket can be attached to the other suction lance and a plug on the associated container, so that the identical plug connector can be used in terms of structure, and only the plugs and sockets must be exchanged. The advantage of this configuration is that the identical plug/socket combination can be used.

For distinguishing more than two containers, in one embodiment different plug/socket combinations are used that are incompatible with each other.

In another aspect of the invention, additional identification features can be attached to the corresponding container, such as a microchip or a transponder, which is activated by a current flow when it is connected to the suction lance and then transmits additional information on the liquid, such as production date, expiration date, filling date, etc., to the machine controller. This transmission can be realized using wireless or wired methods. In this way, the machine can prevent, by appropriate identification of the individual container, the container from being connected again a second time to the suction lance of the machine without permission after it has been refilled with liquid. This represents another safety step against intentional incorrect refilling or use of foreign chemicals in the machine.

With the invention, at least the following advantages are achieved: mix-up of containers is prevented, and leads to shutdown of the machine; the machine prevents unauthorized reuse of containers; the invention can be readily practiced by retrofitting existing machines; the operation of the machine is prevented automatically as long as the corresponding adapter of the plug connector of the container is not connected to the suction lance; and in addition, according to a preferred embodiment of the invention, large amounts of data with additional information about the liquids in the containers can be transmitted to the machine.

Other objects and features will be in part apparent and in part pointed out hereinafter.

### BRIEF DESCRIPTION OF THE FIGURES

FIG. 1 is a schematic diagram of two containers with suction lances and the plug connectors.

### DETAILED DESCRIPTION OF PREFERRED EMBODIMENTS

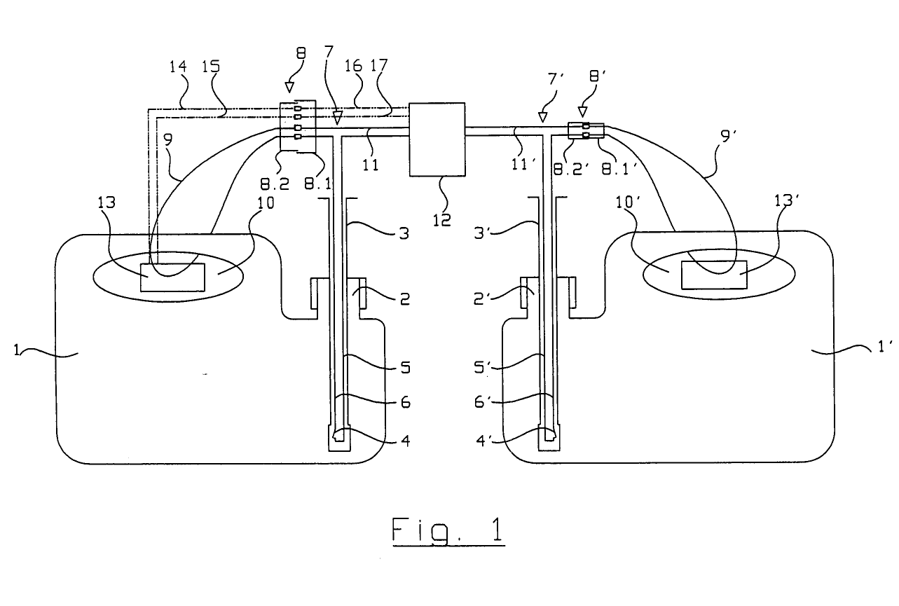

One embodiment of a cleaning and disinfecting machine is shown schematically in FIG. 1. A container 1 for a first liquid has an outlet opening 2, in which a suction lance 3 can be inserted. The suction lance 3 has an electrical float switch 4, which is closed when the container 1 is full and opens when the liquid level in the container 1 has sunk below a certain value. The two contacts of the float switch 4 are connected by means of conductors 5, 6, respectively, of one electrical line to the machine controller 12. However, the conductor 6 is interrupted at a branch point 7 and connected to a component 8.1 of an electrical connector 8. As illustrated, in this embodiment the electrical connector 8 (and 8′) is a plug connector 8 which has been built with two poles and has a plug part 8.1 and a socket part 8.2, but other electrical connectors may be used. One contact of the plug part 8.1 is connected to the conductor 6 and thus to the float switch 4. The other contact of the plug part 8.1 is connected via an electrical line to the machine controller 12. The two contacts of the socket part 8.2 are connected to each other via an electrical jumper line 9, which in turn is permanently joined mechanically to the container 1 (e.g., led through a grip opening 10 of the container). In alternative embodiments, the connections between the connectors and the containers is by other means such as a rope, chain, wire, or cable which does not have an electrical function. If the plug part 8.1 and the socket part 8.2 are connected to each other, then the electrical jumper line 9 connects the two lines 6 and 11 to each other, with the result that the float switch 4 is then correctly connected in terms of its two poles to the controller 12. The plug connector 8 with the electrical jumper line 9 thus acts as an electrical switch or bridge, which creates the connection between the float switch 4 and the controller 12 in the coupled state of the plug connector 8 and breaks this connection in the decoupled state.

Accordingly, FIG. 1 shows a first electrical connector 8 associated with the first exchangeable container 1 and the first suction lance 3. The first electrical connector 8 has a first component 8.2 of the first electrical connector being connected to the first container 1, a second component 8.1 of the first electrical connector being connected to the first suction lance electrical switch 4. There is also the first electrical connector electrical line 9 completing an electrical connection of the first suction lance electrical switch 4 and the controller 12 when the first and second components 8.2 and 8.1 of the first electrical connector are coupled together.

A second container 1′ with outlet opening 2′ is formed identically and is attached via a suction lance 3′ to the float switch 4′ and via electrical lines 5′ and 6′ in the same way to a plug connector 8′ and via an electrical jumper line 9′ to the controller 12. That is, the second electrical connector 8′ is associated with the second exchangeable container 1′ and the second suction lance 3′. The second electrical connector 8′ has a first component 8.1′ of the second electrical connector being connected to the second container 1′, a second component 8.2′ of the second electrical connector being connected to the second suction lance electrical switch 4′, and a second electrical connector electrical line 9′ completing an electrical connection of the second suction lance electrical switch and the controller 12 when the components of the second electrical connector are coupled together.

The connector 8′ is not compatible with the connector 8 of the first container 1. Thus, a uniquely different connector, shown here as plug connector 8 or 8′, is associated with each pair of container 1 or 1′ and suction lance 3 or 3′, so that the suction lance 3 for the first container 1 can only be attached to this container and not to the second container 1′ and vice versa.

Thus, the corresponding container is indirectly identified by the plug connectors 8 and 8′, so that mix-ups are substantially prevented. The controller 12 detects when either of the plug connectors 8, 8′ is in a decoupled state and generates an error signal that prevents operation of the machine.

The different plug connectors 8, 8′ can be realized in various ways. In the simplest case, which is suitable for a machine having only two containers 1, 1′ and two suction lances 3, 3′, the plug part 8.1 for container 1 is attached to the suction lance 3 and the socket part 8.2 is attached to the container 1, while the socket part 8.2′ is attached to the second suction lance 3′ and the plug part 8.1′ is attached to the second container 1′. If someone mistakenly attempts to connect container 1 to the wrong suction lance 3′ and container 1′ to the wrong suction lance 3, the two socket parts and two plug parts could not be coupled to each other. This would prevent the container 1 from being connected to suction lance 3′ and prevent container 1′ from being connected to suction lance 3. In this embodiment, the plug connectors 8, 8′ are incompatible (even if they are identical in design) because of the way the socket parts and plug parts are connected to the containers 1, 1′ and lances 3, 3′.

According to another variant of the invention, which is also suitable for more than two containers and suction lances, the plug parts 8.1, 8.1′ are built to be mechanically different from each other, so that the plug parts 8.1, 8.1′ can be coupled to the corresponding socket parts 8.2, 8.2′, respectively, but cannot be coupled to any of the other socket parts. This can be realized by designing plug parts having different diameters, different shapes, or different arrangement of the contacts to be connected to each other. By extension of the principles set forth above, machines can be designed to use any number of containers concurrently by including a sufficient number of unique plug connectors for each container and suction lance.

In another aspect of the invention, a microchip or transponder 13, 13′ can be assigned to each container 1, 1′, respectively, which is activated by a current flow through the respective jumper line 9, 9′ and transmits data to the controller 12 using wired or wireless methods. This data can contain additional information on the liquid found in the container 1, 1′, the production date, the expiration date, the filling date, the batch number, a container number or the like. In this way, each individual container 1, 1′ can receive an individual identification which is transmitted to the machine controller 12. Thus, refilling of either of the containers 1, 1′ without permission and connecting a second time to the associated suction lance 3, 3′ can also be prevented, because the controller 12 can store the information that the containers 1, 1′ were already connected once to the machine. This represents an additional safety precaution against incorrect refilling or the use of foreign chemicals.

For wired transmission from the microchips 13, 13′ to the controller 12, the connection of the micro chips to the controller can be realized via the plug connectors 8, 8′. As shown in FIG. 1 for microchip 13, for example, the plug connector 8 can be configured with more than two poles, e.g., it can have four poles, with data transmission lines 14 and 15 being connected from the microchip 13 via the plug connector 8 to data transmission lines 16 and 17, which are connected to the controller 12. Alternatively, as shown in FIG. 1 for microchip 13′ the data can be sent to the controller 12 through the electrical jumper line 9′, and electrical lines 5′, 6′, and 11′.

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above methods and products without departing from the scope of the invention, it is intended that all matter contained in the above description and shown in the accompanying drawings shall be interpreted as illustrative and not in a limiting sense.

1. A cleaning and disinfecting machine of the type having multiple containers for containing liquid and suction lances therefor, the machine comprising:
a first exchangeable container containing a first liquid and a second exchangeable container containing a second liquid, the second liquid being different from the first liquid;
a first suction lance operable to suck the first liquid out of the first container and a second suction lance operable to suck the second liquid out of the second container, the first suction lance comprising a first suction lance electrical switch for connection to a machine controller via a first electrical line and the second suction lance comprising a second suction lance electrical switch for connection to the machine controller via a second electrical line;
a first electrical connector associated with the first exchangeable container and the first suction lance, the first electrical connector having a first component of the first electrical connector being connected to the first container, a second component of the first electrical connector being connected to the first suction lance electrical switch, and a first electrical connector electrical line completing an electrical connection of the first suction lance electrical switch and the controller when the first and second components of the first electrical connector are coupled together; and
a second electrical connector associated with the second exchangeable container and the second suction lance, the second electrical connector having a first component of the second electrical connector being connected to the second container, a second component of the second electrical connector being connected to the second suction lance electrical switch, and a second electrical connector electrical line completing an electrical connection of the second suction lance electrical switch and the controller when the components of the second electrical connector are coupled together.

2. The cleaning and disinfecting machine according to claim 1, wherein the first component of the first electrical connector which is connected to the first container is a socket part, the first component of the second electrical connector which is connected to the second container is a plug part, the second component of the first electrical connector is a plug part, and the second component of the second electrical connector is a socket part.

3. The cleaning and disinfecting machine according to claim 1, wherein the first electrical connector has a geometry which is distinct from a geometry of the second electrical connector so that the first and second components of the first electrical connector cannot be connected to first component of the second electrical connector and so that the first and second components of the first electrical connector cannot be connected to second component of the second electrical connector.

4. The cleaning and disinfecting machine according to claim 3, wherein the first and second components of the first electrical connector differ from the first and second components of the second electrical connector with respect to at least one of the following: diameter, shape, and arrangement of contacts.

5. The cleaning and disinfecting machine according to claim 1, wherein the first electrical connector electrical line and second electrical connector electrical line are jumper lines which are permanently connected mechanically to the respective containers.

6. The cleaning and disinfecting machine according to claim 3, wherein the first electrical connector electrical line and second electrical connector electrical line are jumper lines which are permanently connected mechanically to the respective containers.

7. The cleaning and disinfecting machine according to claim 1, further comprising a first microchip assigned to the first container and activated by coupling the first and second components of the first electrical connector, the first microchip being operable to transmit information about the first container to the controller.

8. The cleaning and disinfecting machine according to claim 7, wherein the first microchip is operable to transmit the information to the controller through the first electrical connector electrical line.

9. The cleaning and disinfecting machine according to claim 7, wherein the first microchip is operable to transmit the information to the controller through a separate data transmission line.

10. The cleaning and disinfecting machine according to claim 7, wherein the first microchip is operable to transmit the information to the controller using a wireless method by means of electromagnetic waves.

11. The cleaning and disinfecting machine according to claim 7, further comprising a second microchip assigned to the second container and activated by coupling the first and second components of the second electrical connector, the second microchip being operable to transmit information about the second container to the controller.

12. The cleaning and disinfecting machine according to claim 11, wherein the first and second microchips are operable to transmit the information to the controller through the first and second electrical connector electrical lines.

13. The cleaning and disinfecting machine according to claim 11, wherein the first and second microchips are operable to transmit the information to the controller through separate data transmission lines.

14. The cleaning and disinfecting machine according to claim 11, wherein the first and second microchips are operable to transmit the information to the controller using a wireless method by means of electromagnetic waves.

15. The cleaning and disinfecting machine according to claim 7, wherein the microchip is a transponder.

16. The cleaning and disinfecting machine according to claim 7, wherein the controller has a memory operable to store data transmitted from the microchip and to prevent operation of the machine if a container that has previously been connected to the controller is reconnected.

17. The cleaning and disinfecting machine according to claim 1 wherein the first component of the first electrical connector is incompatible with the second component of the second electrical connector.

18. The cleaning and disinfecting machine according to claim 1 wherein the first component of the second electrical connector is incompatible with the second component of the first electrical connector

19. The cleaning and disinfecting machine according to claim 1 wherein the first component of the first electrical connector is incompatible with the second component of the second electrical connector and the first component of the second electrical connector is incompatible with the second component of the first electrical connector.

20. The cleaning and disinfecting machine according to claim 1, wherein the first and second components of the first electrical connector are incompatible with the first and second components of the second electrical connector.

21. A cleaning and disinfecting machine of the type having multiple containers for containing liquid and suction lances therefor, the machine comprising:
a first exchangeable container containing a first liquid and a second exchangeable container containing a second liquid, the second liquid being different from the first liquid;
a first suction lance operable to suck the first liquid out of the first container and a second suction lance operable to suck the second liquid out of the second container, the first suction lance comprising a first suction lance electrical switch connected to a machine controller via a first electrical line and the second suction lance comprising a second suction lance electrical switch connected to the machine controller via a second electrical line;
a first plug connector associated with the first exchangeable container and the first suction lance, the first plug connector having a first component of the first plug connector being connected to the first container via a first electrical jumper line and a second component of the first plug connector being connected to the first suction lance electrical switch, the first electrical jumper line completing electrical connection of the first suction lance electrical switch and the controller when the first and second components of the first plug connector are coupled together; and
a second plug connector associated with the second exchangeable container and the second suction lance, the second plug connector having a first component of the second plug connector being connected to the second container via a second electrical jumper line and a second component of the second plug connector being connected to the second suction lance electrical switch, the second electrical jumper line completing electrical connection of the second suction lance electrical switch and the controller when the components of the second plug connector are coupled together.
